(19) United States
(12) Patent Application Publication
(10) Pub. No.: US 2005/0197700 A1
(43) Pub. Date: Sep. 8, 2005

(54) Facet joint prosthesis and method of replacing a facet joint

(76) Inventors: Frank H. Boehm JR., Utica, NY (US); Benedetta Delorenzo Melnick, Rome, NY (US)

Correspondence Address: DILWORTH & BARRESE, LLP, 333 EARLE OVINGTON BLVD., UNIONDALE, NY, 11553, US

(21) Appl. No.: 11/061,802

(22) Filed: Feb. 18, 2005

Related U.S. Application Data
(60) Provisional application No. 60/545,449, filed on Feb. 18, 2004.

(51) Int. Cl. A61F 2/44

(52) U.S. Cl. 623/17.11; 606/61

(57) ABSTRACT

A prosthetic device for replacing a facet joint in the spine of a patient is provided. The prosthetic device includes an anchoring member and a plate member rotatably and pivotably connected to each other by a ball and socket mechanism. The anchoring member is preferably in the form of a bone screw for placement into the pedicle/facet intersection of a vertebrae, and the plate member is provided with securing holes for securing the plate member to an adjacent vertebrae. A minimally invasive, percutaneous surgical method is also provided for replacing the facet joint in the spine, and a kit for performing the surgical method is also provided.

### PRIORITY

This application claims priority to a provisional application entitled “PERCUTANEOUS FACET JOINT PROSTHESIS”, filed in the U.S. Patent and Trademark Office on Feb. 18, 2004 and assigned Ser. No. 60/545,449, the contents of which are incorporated herein by reference.

### BACKGROUND

1. Field of the Invention

The present invention relates generally to prosthetic devices, and in particular to a prosthetic device for replacing a facet joint in the spine of a patient, and to a method for replacing a facet joint in the patient's spine.

2. Description of the Related Art

Degenerative diseases of the spine are a major cause of morbidity in the United States and around the world. Back pain is one of the leading causes of disability in this country, and costs billions of dollars each year to both patients and their insurance companies. Back pain is the most common reason for patients to consult doctors in this country.

The human spine supports and stabilizes a person, and is formed by bony structures known as the vertebra, which are separated and cushioned by discs which are located between each of the vertebrae. The vertebra are grouped by location, i.e., lumbar, thoracic, and cervical.

There are many degenerative diseases of the spine, and may involve diseases of the discs, the vertebra, or neurological diseases of the spinal cord itself and the many nerves that extend therefrom. In response to the overwhelming problem of back pain, many strategies have been developed to address specific diseases and specific pain issues.

To address degenerative disc disease, techniques have been developed to replace the diseased disc with an artificial disc. While there have been reports of success with these procedures, recently the long-term effects have been called into question. A multi-center clinical study has been established to evaluate the utility and safety of disc replacement, as well as the long-term benefits of total disc replacement.

The discs of the spinal column are not isolated anatomical structures, but are in reality components of a larger system known as a vertebral motion segment. The vertebral motion segment is comprised of two adjacent vertebrae, with the intervertebral disc located between the discs on the anterior side of the spinal column, and the facet joints and processes, such as the spinus process and transverse processes located on the posterior side of the spinal column. Muscles and ligaments further contribute to the motion segment, and these components of the vertebral motion segment work in concert with each other. In particular, the disc and facet joints at each disc level contribute to both the stability and flexibility of the spine. It is generally thought that the discs govern anteriorly directed bending motions and forward flexion motion, in addition to their role in stabilizing the spine. Conversely, the facets act to limit extension and to stabilize translational motion. The discs and facet joints apparently work together in reciprocal fashion, each stabilizing the action of the other. Therefore, many times diseased discs are often associated with disease of the corresponding facet joints. Although a consensus as to the exact role of the facet joint has not been established in the medical community, it is clear that their role has been underestimated.

There has been increased interest in replacement of the facet joints, both as a procedure performed in conjunction with disc replacement, and as stand alone procedure for removing diseased or arthritic facet joints as a means for pain reduction. As a result, many techniques and prostheses have been developed in recent years. These techniques typically require an open type procedure, in which a large incision is made to access the facet joint to be replaced. Consequently, trauma to the patient is increased, and the hospital stay and recovery period is likewise increased.

In addition, the facet joint prostheses that have recently been developed are highly complex with multiple connection points, and require implantation only by an open surgical procedure through a large incision or multiple large incisions to provide the necessary access. These prosthetic devices also appear to be size specific, in that they would need to be custom sized for patients of different height and bone structure. This, of course, would necessarily increase costs, as multiple prosthetic devices would need to be present during the surgery, and the time required for the surgery would also increase as the surgeon attempted to fit the proper device for the patient.

Therefore, a need exists for a facet joint prosthetic device that is relatively universal in its applicability to patients of all sizes. A need also exists for a surgical method for replacing facet joints in a minimally invasive manner, to reduce trauma to the patient and to reduce the recovery time of the post-operative patient.

### SUMMARY

The present invention has been made in view of the above problems, and the present invention provides a prosthetic device for replacing a facet joint in a patient's spine. The present invention also provides a minimally invasive method for replacing a facet joint in the spine. A kit for use by the surgeon performing the minimally invasive surgical method is also provided by the present invention.

In accordance with one aspect of the present invention, the prosthetic device includes a plate member and anchoring member joined to each other about a pivot point to facilitate both rotational movement of the plate member with respect to the anchoring member as well as pivoting movement of the plate member with respect to the anchoring member.

The anchoring member is preferably in the form of a screw, for anchoring the prosthetic device to one of the inferior or superior vertebrae of the facet joint to be replaced. The plate member has a securing hole for a bone screw, to secure the plate member to the other vertebrae adjacent to the vertebrae to which the anchoring screw is attached. The anchoring member terminates in a ball, which along with a cup-shaped holder formed on an edge of the plate member, defines a ball and socket mechanism which permits the rotational and pivoting movement of the prosthetic device. Of course, it is contemplated that the ball may be located on the plate member and the cup on the anchoring member, if desired. The cup has an opening slot on one side, which is preferably slightly larger than the diameter of the shaft of the screw of the anchor member, to permit the plate member to pivot with respect to the anchor member. The length of the slot determines the range of pivoting movement, as well as the position of the implanted prosthetic with respect to the vertical axis of the patient's body. The range of pivoting movement is determined by the location of the facet joint to be replaced, i.e. whether the joint is in the lumbar, thoracic or cervical region of the spine. For example, greater range is required in the lumbar region, and the direction of movement with respect to the vertical axis of the patient's body is also different in the lumbar region than in the thoracic region, due to the anatomical structure of the spine.

In accordance with another aspect of the present invention, a minimally invasive method for replacing a facet joint in the spine of a patient is provided, in which the entire surgical procedure is performed through a small incision in the patient's back. The method includes locating the vertebra where the facet joints are to be replaced and making a small incision in the skin. A guide needle having an outer diameter approximately the same size as a length of the incision is inserted through the incision to a position adjacent the facetjoint on one side of the spinal column. A dilator is then passed over the guide needle to the location of the facet joint to expand the passageway to the facet joint. As needed, additional dilators, each having an inner dilator slightly larger than the outer diameter of a previous dilator, are passed over the previous dilator to enlarge the passageway to a desired size, preferably to a size sufficient to accommodate passage of a facet joint prosthesis. Once the size of the passageway is sufficient, the guide needle and all inner dilators are removed from the outermost dilator, which is left in place to provide the working channel. A cutting tool is passed through the outermost dilator to remove the facet joint, i.e. a portion of the inferior facet of the upper (or superior) vertebrae and a portion of the superior facet of the adjacent lower (or inferior) vertebrae are removed. After the tool and bone fragments are removed from the working channel, the prosthesis is passed to the site. The prosthesis is secured to the lower vertebrae at the location of the removed superior facet, and then is secured upper vertebrae at the location of the removed inferior facet, although the order of securement depends on the prosthesis and the surgeon's choice. Once the prosthesis is secured, the dilator is removed, and the incision is closed. Preferably, the procedure is then repeated to replace the facet joint on the opposite side of the spinal column of the same pair of vertebra.

In addition, a dilator/cannula is provided for facilitating access to the facet joint to be replaced. The dilator has a somewhat oval shape, which facilitates passage through the tissues and muscles of the patient to reduce trauma to the patient by separating, rather than cutting or tearing the tissues and muscles. The oval cross-section defines an x-axis and y-axis, which are dimensioned in a ratio of 2:1, preferably 2:1.5.

A kit for performing a minimally invasive percutaneous facet joint replacement is also provided, which includes at least two prosthetic devices for replacing the facet joints on both sides of a pair of vertebra, a guide needle, at least one dilator to create a working channel to the facet joint to be replaced, and a tool for securing the prosthetic devices to the vertebra. A cutting tool for removing the inferior and superior facets of the joint may also be provided.

### BRIEF DESCRIPTION OF THE DRAWINGS

The above and other features and advantages of the present invention will become more readily apparent from the following detailed description of preferred embodiments of the invention, taken in conjunction with the accompanying drawings, in which:

FIGS. 1A-1D illustrate front, top, side and rear elevation views, respectively, of a preferred embodiment of the facet joint prosthetic device of the present invention;

FIG. 2 illustrates an exploded perspective view of the facet joint prosthetic device of FIG. 1;

FIG. 3 illustrates a perspective view of the prosthetic device of FIG. 1 in which the plate member is pivoted with respect to the anchoring member;

FIG. 4 illustrates an alternate embodiment of the facet joint prosthetic device of the present invention;

FIG. 5 is a posterior view of a pair of lumbar vertebrae;

FIG. 6 is a posterior view of the pair of lumbar vertebrae with a guide needle having been placed adjacent to a facet joint to be replaced;

FIG. 7 is a posterior view of the pair of lumbar vertebrae showing a series of dilators, each successive dilator being slightly larger than the previous dilator, passed over the guide needle during creation of a working channel;

FIG. 8 is a posterior view of the pair of vertebrae with the working channel established and the inner dilators and guide needle removed;

FIG. 9 is a posterior view of the pair of vertebrae after a cutting tool has been inserted through the working channel to remove the facets of the vertebrae, which make up the facet joint;

FIG. 10 is a posterior view showing the prosthetic device of FIG. 1 being passed through the working channel dilator with the use of an insertion/driver tool;

FIG. 11 illustrates a partial cut-away view of the plate member of the prosthetic device with the insertion/driver tool connected to the prosthetic device;

FIG. 12 is a posterior view of the pair of vertebrae with the anchoring member of the prosthetic device having been secured to the lower vertebrae and with the plate member pivoted towards the upper vertebrae, and secured thereto by screws;

FIG. 13 is a transaxial view of the prosthetic device in its final secured position, with the upper vertebrae omitted for clarity;

FIG. 14 illustrates another embodiment of the facet joint prosthetic device of the present invention;

FIGS. 15A-15B illustrate a perspective view and end view, respectively, of a dilator of the present invention; and

FIG. 16 illustrates a kit for percutaneously replacing facet joints according to the present invention.

### DETAILED DESCRIPTION

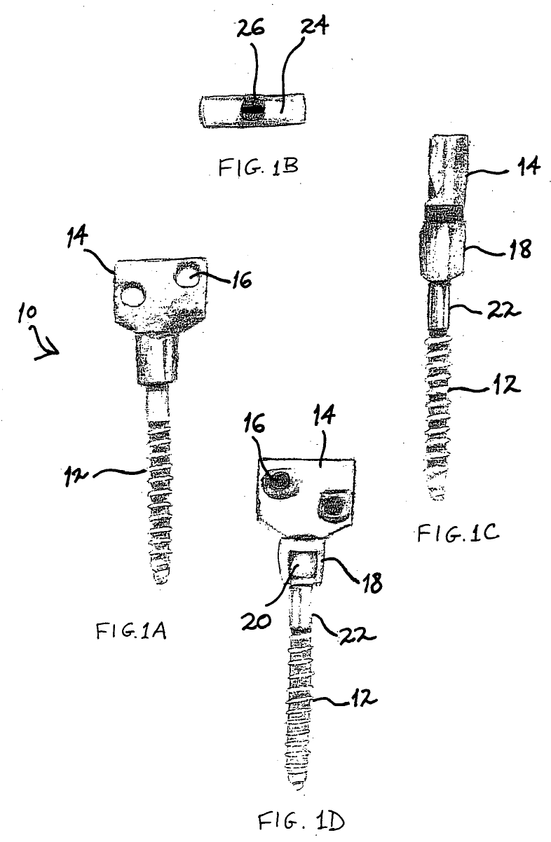

Referring now to the drawings, in which like reference numerals identify similar or identical elements throughout the several views, and in particular to FIGS. 1-3, there is shown the facet joint prosthetic device of the present invention. Preferably, prosthetic device 10 is comprised of an anchoring member 12, which is both rotatably and pivotably attached to a plate member 14. Anchoring member 12 is preferably a bone screw, having threads of sufficient pitch and thickness to allow anchoring member 12 to be self-tapping into the bone of the vertebrae to which it is to be secured. Although anchoring member is preferably a bone screw, other anchoring means for bone securement are also contemplated. Preferably, prosthetic device 10 is made of any non-absorbable, bio-compatible material of the prosthetic arts, such as titanium, stainless steel, porcelain, or a combination of these materials.

Plate member 14 is preferably provided with a pair of securing holes 16, through which bone screws 34 may be placed to secure the plate member 14 to the vertebrae to which it is to be secured. While self-tapping screws are preferred, other securement means are also contemplated, and any number of holes may be provided.

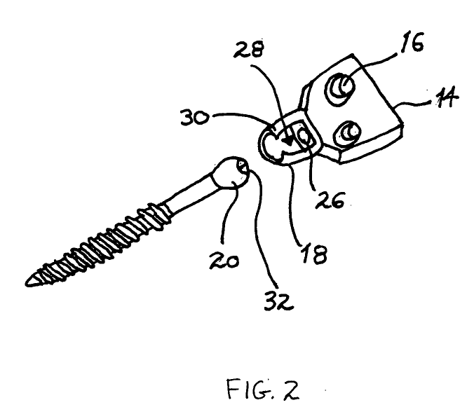

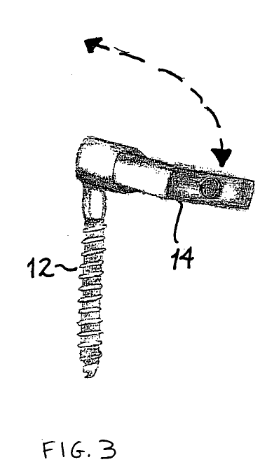

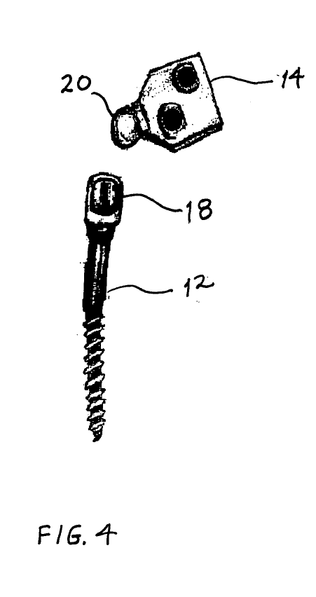

Plate member 14 is joined to anchoring member 12 in a preferred embodiment by a ball and socket mechanism, which permits rotation and pivoting movement between the two. Plate member 14 is provided with a cup 18 which holds a ball 20 formed on an end of the shaft 22 of anchoring member 12. As seen in FIG. 2, cup 18 is provided with a slot 28, which as seen in FIG. 3, facilitates the pivoting movement between plate member 14 and anchoring member 12. The plate member 14 pivots at least 45° from the vertical axis of the anchoring member 12, and may pivot at least 90° in each direction from the vertical axis, depending on the length of slot 28, and whether slot 28 is provided on one side of cup 18 or both sides. The ends of the slot are pinched at 30, to capture and hold the ball 20 of anchoring member 12. Of course, the cup 18 may be provided on the shaft 22 of anchoring member 12 and the ball 20 may be provided on plate member 14, as shown in FIG. 4.

Plate member 14 is provided with a bore 26 that runs from top surface 24 to the interior of cup 18. The top of ball 20 is provided with a driver slot 32, which may accommodate the tip of an insertion/driver tool to secure the prosthetic device 10 to a vertebrae, as will be described below. The driver slot may be a flat head, Phillips, hex-head, or any other type of driver slot.

While plate member 14 is shown as having flat surfaces for placement against the vertebrae at the location of the removed facet, it is also contemplated that the surface which faces the bone is scored to facilitate bone growth.

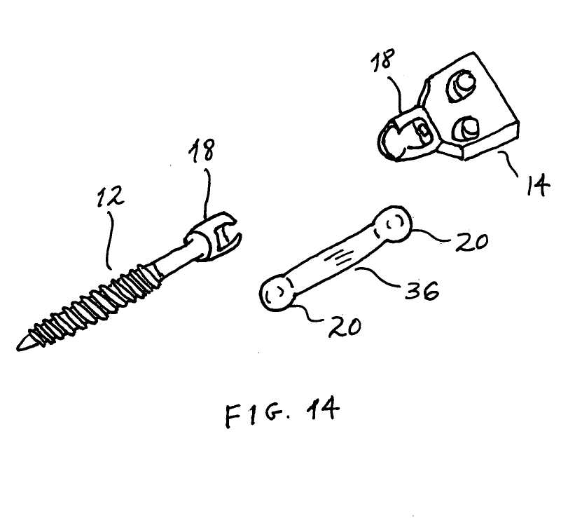

In another embodiment, as shown in FIG. 14, both the anchoring member 12 and the plate member 14 may be provided with cups 18, and after being fixed to their respective vertebrae, are joined by a linkage 36, which is provided with a shaft 38 which terminates in a ball 20 on each end. The balls 20 on either end of the linkage 36 are snap-fit into the cups on each of the anchoring member 12 and plate member 14. Of course, each of anchoring member 12 and plate member 14 may be provided with balls 20, and linkage 36 provided at each end with cups 18, or anchoring member 12 may have a ball 20, to link to a cup 18 on one end of linkage 36, while the other end of linkage 36 is provided with a ball 20 to link to cup 18 on plate member 14. In addition, anchoring member 12 may be provided with a cup 18 to link to a ball 20 on linkage 36, while the other end of linkage 36 is provided with a cup 18 to link to a ball 20 on plate member 14. Using this linkage arrangement, it is also contemplated that plate member 14 can be replaced with another anchoring member 12 for direct securement to the upper vertebrae.

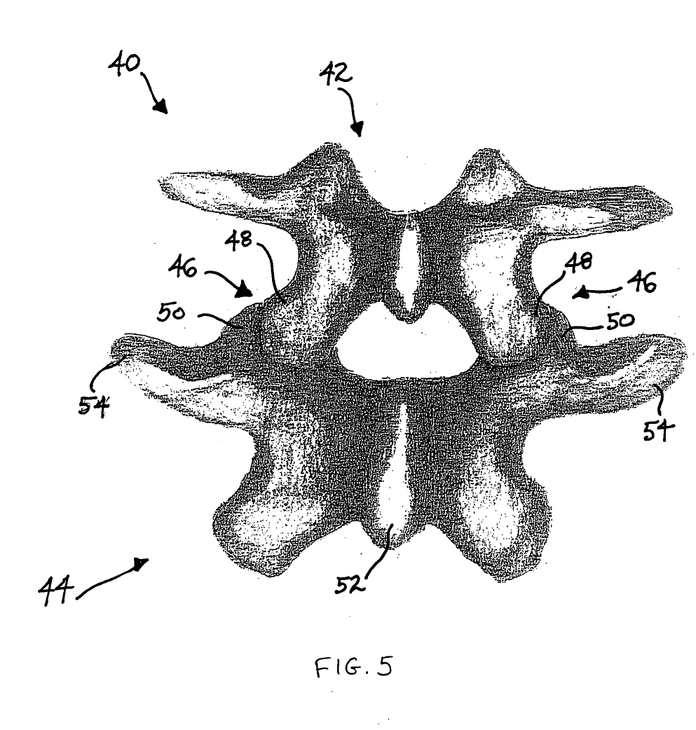

For purposes of explanation, FIG. 5 shows a posterior view of a pair of lumbar vertebrae, to which the present invention is applicable. While lumbar vertebra are shown, the invention is of course applicable to any adjacent vertebra. The pair of vertebrae 40 is comprised of superior (upper) vertebrae 42 and inferior (lower) vertebrae 44. The facet joints 46 between the pair 40 are comprised of inferior facet 48 of superior vertebrae 42, and superior facet 50 of inferior vertebrae 44. The vertebra are also comprised of spinus process 52 and transverse processes 54. Hereinafter, for ease of understanding, superior vertebrae 42 will simply be referred to as upper vertebrae 42, and inferior vertebrae 44 will be referred to as lower vertebrae 44.

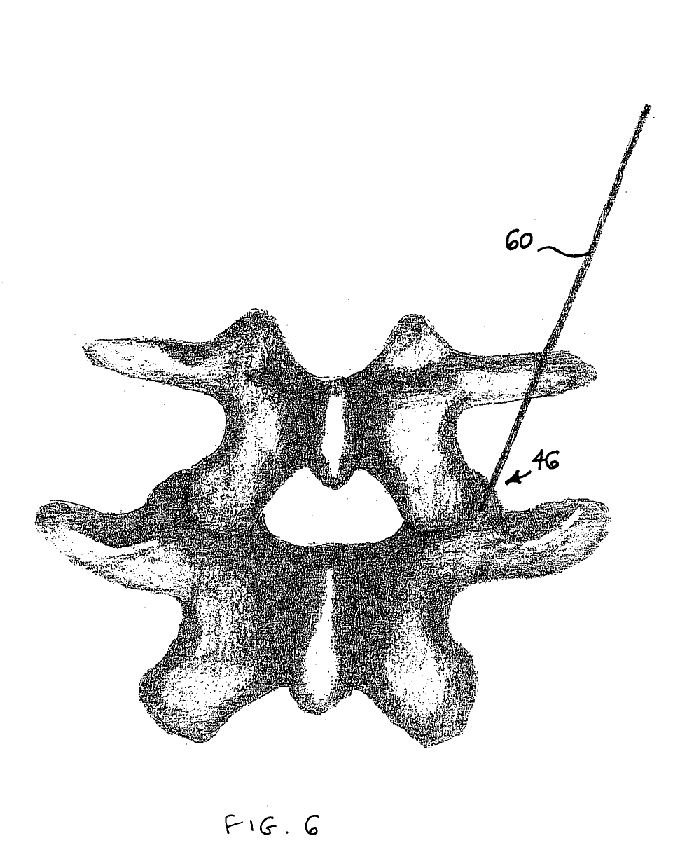

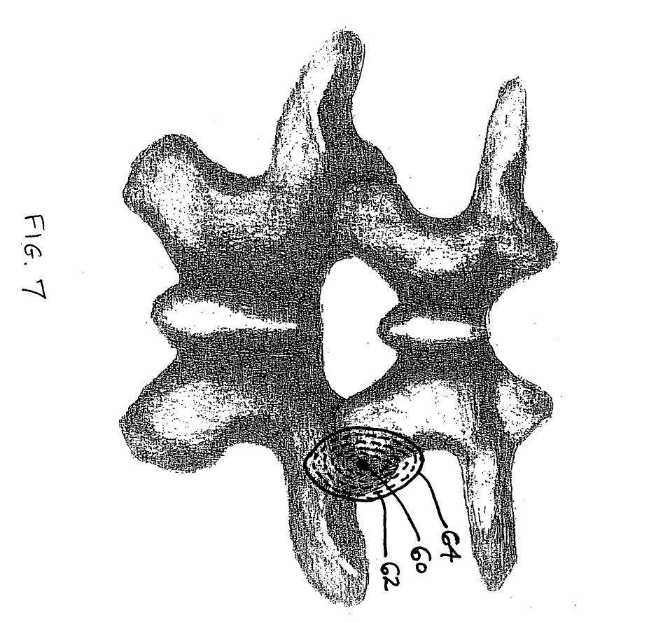

Referring now to FIGS. 6-14, the method of replacing a facet joint according to the present invention will now be described. After the surgeon locates the pair of vertebrae 40 whose facet joints are to be replaced, which is typically done fluoroscopically or by another imaging procedure, a small incision is made through the patient's skin and a guide needle 60 is passed to the vicinity of the facet joint 46. The insertion of guide needle 60 is preferably done under fluoroscopy. It is noted that FIG. 6 shows guide needle 60 at an exaggerated angle; this is shown for purposes of facilitating understanding of the insertion of guide needle 60. In reality, guide needle 60 is inserted generally perpendicular to the facet joint, although it may be at angle to the plane of the patient's skin, particularly in the lumbar region, due to the natural anatomic curvature of the spine.

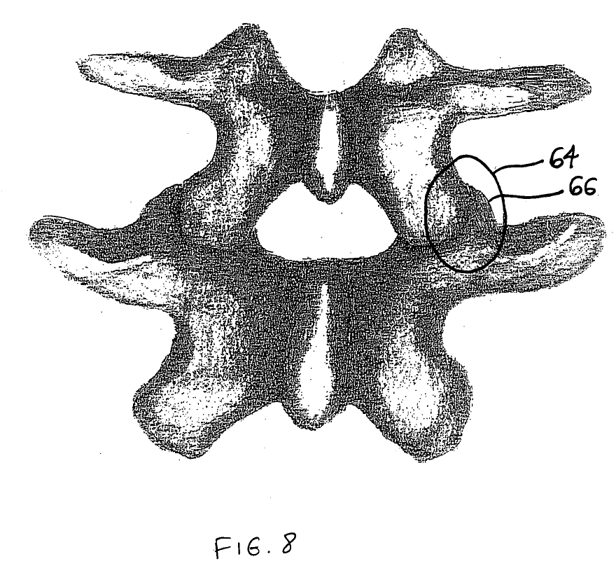

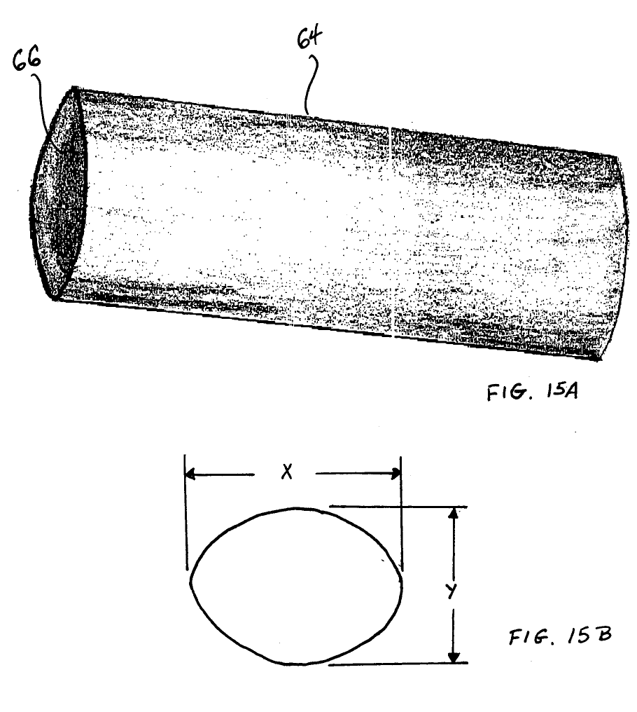

Once guide needle 60 is in place, a series of dilators 62, 64 are passed over the guide needle 60 to expand the incision and to create a working channel for the surgeon to replace the diseased facet joint. Preferably, as seen in FIGS. 15A and 15B, the dilators have a somewhat oval shape, which facilitates passage through the patient's muscles and tissue and minimizes trauma by separating, rather than cutting, the muscles and tissue. The ratio between the length of the dilator (taken along the x-axis in FIG. 15B) and the width of the dilator (taken along the y-axis of FIG. 15B) is at least 2 to 2, and is preferably 2 to 1.5. The series of dilators are successively larger, where the inner diameter, in both the x-direction and the y-direction, of a next dilator is slightly larger than the outer diameter of the previous dilator, while the ratio between the width and length remains constant. This ensures a gentle enlargement of the incision and the pathway through the muscles, to prevent tearing. Once a working channel 66 (FIG. 8) of a desired size is established, outermost dilator 64 is left in place and each of the inner dilators 62, and guide needle 60, are removed.

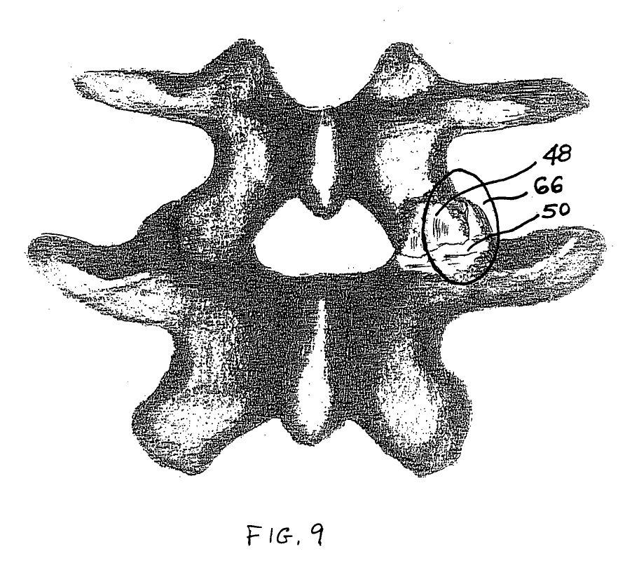

The facet joint 46 is then removed by a cutting tool, which preferably removes the diseased portion of both the inferior facet 48 and the superior facet 50, and creates a small space between the facets. Preferably the surface of the remaining portion of the inferior facet 48 is prepared to accommodate the flat surface of plate member 14, as seen in FIG. 9.

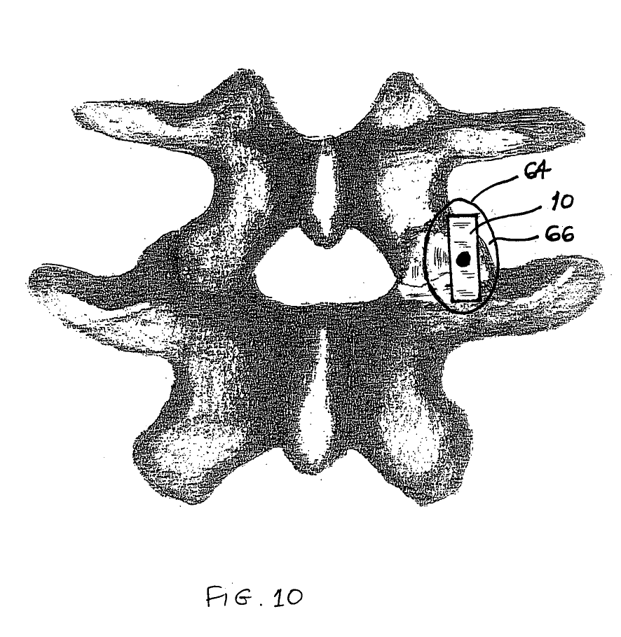

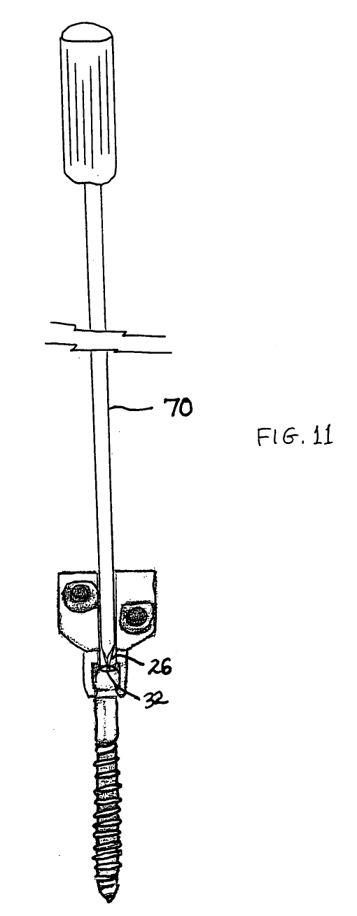

After the cutting tool and bone fragments are removed through the working channel, the facet joint prosthetic device 10 is prepared for introduction to the site. As seen in FIG. 11, an insertion/driver tool 70 is inserted through the bore 26 in plate member 14 so that the tip of the driver contacts the driver slot 32 on the ball 20 anchoring member 12. In the embodiment of FIG. 4, tool 70 passes through the bore 26 which extends through ball 20 on plate member 14, and contacts the driver slot 32 at the base of cup 18 at the end of shaft 22 of anchoring member 12. Tool 70 holds prosthetic device 10 to allow the surgeon to pass prosthetic device 10 through the working channel, as seen in FIG. 10.

Referring to FIG. 10, once the prosthetic device 10 is passed through the working channel, anchoring member 12 is screwed into pedicle/facet intersection of the lower vertebrae 44. Plate member 14, being rotatably connected to anchoring member 12, is maintained in the position shown in FIG. 10 while anchoring member 12 is screwed into the lower vertebrae 44. This enables a smaller outermost dilator 64 to be used, and allows a more accurate placement of the plate member 14 on the upper vertebrae 42, since the pivoting and rotating movement of the plate member 14 with respect to the anchoring member 12 affords greater flexibility for the surgeon.

While it has been described that the anchoring member 12 is secured to the lower vertebrae and the plate member 14 is secured to the upper vertebrae, it is also contemplated that the anchoring member may be secured to the upper vertebrae while the plate member 14 is secured to the lower vertebrae. This arrangement would be particularly useful in the cervical spine.

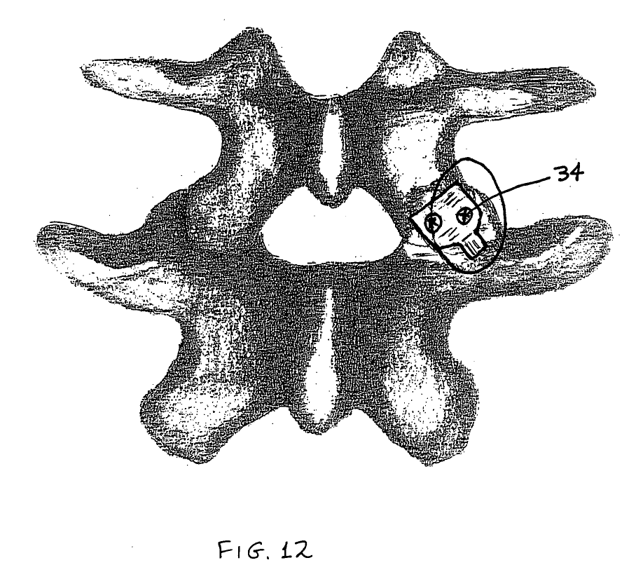

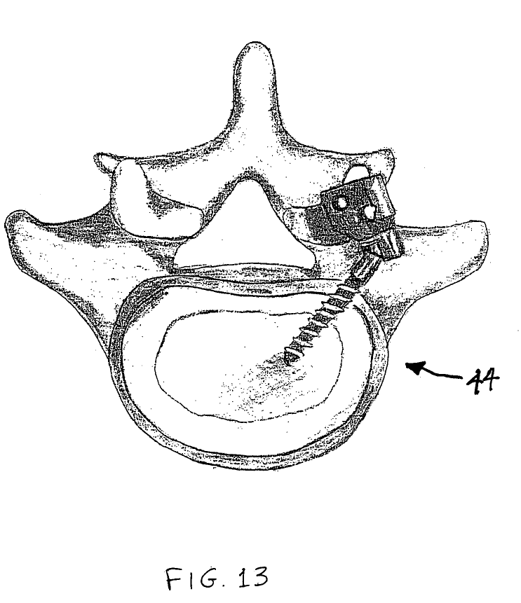

Once the anchoring member 12 is fully inserted (FIG. 13), the position of the plate member 14 is adjusted, and the plate member 14 is pivoted to the position shown in FIG. 12. Screws 34 are inserted into securing holes 16 and screwed into the upper vertebrae to secure the plate member 14.

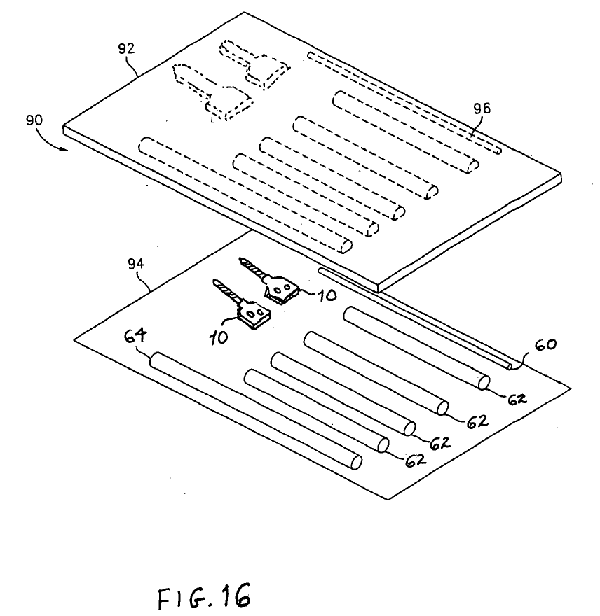

FIG. 16 illustrates a kit for percutaneously replacing a facet joint in a patient's spine according to the present invention. Kit 90 comprises a package having a top cover 92 and bottom cover 94, where top cover '92 is preferably of plastic having depressions or indentations 96 for holding the instruments packaged therein. Packaged in kit 90 are preferably at least two facet joint prosthetic devices 10, a plurality of dilators 62, 64, and a guide needle 60. Insertion/driver tool 70.may also be provided in kit 90. Kit 90 is preferably sterilized.

While the invention has been shown and described with reference to certain preferred embodiments, it will be understood by those skilled in the art that various changes and modifications in form and detail may be made therein without departing from the spirit and scope of the invention, as defined by the appended claims.

1. A prosthetic device for replacing a facet joint between adjacent vertebra in a spine, comprising:
an anchor member for securing the prosthetic device to a first vertebrae; and
a plate member pivotably connected to the anchor member for attachment to a second vertebrae adjacent to the first vertebrae, the plate member being pivotable in at least 45° from a longitudinal axis of the anchor member.

2. The device of claim 1, wherein the anchoring member comprises a bone screw.

3. The device of claim 1, wherein the anchor member and plate member are connected by a ball and socket mechanism.

4. The device of claim 3, wherein the ball is provided on the anchoring member, and a cup comprising the socket is provided on the plate member.

5. The device of claim 1, wherein the anchoring member and plate member are connected by a linkage.

6. The device of claim 5, wherein the linkage is connected to each of the anchoring member and the plate member by a ball and socket mechanism.

7. The device of claim 1, wherein the plate member is rotatable about the anchoring member at least 360°.

8. A facet joint prosthesis, comprising:
an anchor member; and
a plate member connected to the anchor member to permit at least 360° rotation movement of the plate member with respect to the anchor member about a first axis, and to permit pivoting movement of the plate with respect to the anchor member about a second axis, wherein the first axis is perpendicular to the second axis.

9. A prosthetic device for replacing a facet joint between an inferior vertebrae and a superior vertebrae, wherein at least a portion of the superior facet of the inferior vertebrae and a portion of the inferior facet of the superior vertebrae have been removed, the prosthetic device comprising:
an anchor member for securing the prosthetic device to one of the inferior vertebrae and the superior vertebrae at a location of the removed facet; and
a plate member for attachment to the other of the inferior vertebrae and the superior vertebrae at a location of the removed facet, the plate member being pivotable and rotatable with respect to the anchor member.

10. The device of claim 9, wherein the anchoring member is secured to the inferior vertebrae at a pedicle/facet intersection.

11. The device of claim 10, wherein the plate member is secured to the superior vertebrae at the location of the removed inferior facet.

12. The device of claim 9, wherein the plate member is rotatable about a vertical axis of the anchoring member, and pivotable about a second axis perpendicular to the vertical axis.

13. A method for replacing a facet joint in a spine, comprising the steps of:
accessing a facet joint between an inferior vertebrae and a superior vertebrae adjacent to the inferior vertebrae;
removing a portion of the inferior facet of the superior vertebrae;
removing a portion of the superior facet of the inferior vertebrae adjacent to the removed inferior facet;
securing a prosthetic device to the inferior vertebrae at a location corresponding to the removed superior facet portion, and to the superior vertebrae at a location corresponding to the removed inferior facet portion, wherein a part of the prosthetic device which is secured to the inferior vertebrae is pivotably connected to a part of the prosthetic device which is secured to the superior vertebrae.

14. The method of claim 13, wherein the step of accessing comprises inserting at least one dilator to create a working channel to the facet joint to be replaced.

15. The method of claim 14, wherein the removing steps and the securing step are performed through the working channel.

16. The method of claim 13, wherein the parts of the prosthetic device are connected to each other to permit pivoting and rotational movement between the parts.

17. A method of replacing a facet joint in a spine, comprising the steps of:
accessing a facet joint between an inferior vertebrae and a superior vertebrae adjacent to the inferior vertebrae;
removing a portion of the inferior facet of the superior vertebrae;
removing a portion of the superior facet of the inferior vertebrae adjacent to the removed inferior facet;
securing an anchor member of a prosthetic device to a pedicle of the inferior vertebrae, the prosthetic device having a plate member pivotably connected to the anchor member;
securing the plate member to the superior vertebrae at a location corresponding to the removed inferior facet.

18. A method of percutaneously replacing a facet joint in a spine of a patient, the facet joint to be replaced being located by an imaging process and consisting of an inferior facet of a superior vertebrae and a superior facet of an adjacent inferior vertebrae, the method comprising the steps of:
making an incision;
inserting a guide needle through the incision to a position adjacent the facet joint to be replaced;
inserting at least one dilator over the guide needle and advancing the at least one dilator to a position adjacent the facet joint to create a working channel;
removing the guide needle from the at least one dilator;
inserting a tool to remove at least a portion of the inferior facet and at least a portion of the superior facet through the outermost dilator;
removing the tool;
removing the portions of the inferior and superior facets through the dilator;
inserting a prosthetic device to the location of the removed facet joint;
securing a first part of the prosthetic device to one of the inferior vertebrae and superior vertebrae;
securing a second part of the prosthetic device to the other of the inferior and superior vertebrae at a location corresponding to the removed facet, the second part of the prosthetic device being pivotably connected to the first part;
removing the dilator; and
closing the incision.

19. The method of claim 18, wherein the guide needle has a diameter substantially the same as a length of the incision.

20. The method of claim 18, further comprising a plurality of dilators, the inner diameter of each successive dilator being slightly larger than an outer diameter of a previous dilator.

21. The method of claim 20, further comprising the step of, after creating the working channel, removing each previous dilator while maintaining an outermost dilator in the incision to define the working channel.

22. A method for percutaneously replacing a facet joint in a spine, the facet joint including an inferior facet and a superior facet of adjacent vertebra, the method comprising:
locating the facet joint to be replaced by an imaging process;
making an incision;
inserting a guide needle to the location of the facet joint through the incision;
inserting a first dilator over the guide needle to the location of the facet joint to expand the incision;
inserting a plurality of dilators over the first dilator to the location of the facet joint, each successive dilator having an inner diameter being slightly larger than the outer diameter of the previous dilator to expand the incision to define a working channel defined by an outermost dilator;
removing each dilator and the guide wire while maintaining the outermost dilator in the incision with an end of the outermost dilator being adjacent the facet joint;
removing at least a portion of the inferior and superior facets of the facet joint;
inserting a prosthesis device to the location of the facet joint;
securing a first end of the prosthesis device to one vertebrae;
securing a second end of the prosthetic device to an adjacent vertebrae at a location corresponding to the removed facet, the first end of the prosthetic device being pivotably connected to the second end of the prosthetic device;
removing the outermost dilator; and
closing the incision.

23. A kit for replacing a facet joint in a spine, comprising:
a guide needle;
a plurality of dilators for creating a working channel from the exterior of a patient to the location of the facet joint, the plurality of dilators including a first dilator having an inner diameter being larger than an outer diameter of the guide needle, the remaining dilators successively increasing in diameter whereby a successive dilator has an inner diameter that is slightly larger than an outer diameter of a previous dilator;
at least one prosthetic device having a first part for securement to an inferior vertebrae and a second part for securement to a superior vertebrae, the first part of the prosthetic device being pivotably connected to the second part; and
a package for enclosing each of the guide needle, the plurality of dilators, and the at least one prosthetic device.

24. The kit of claim 23, further comprising an insertion/driver tool for securing the at least one prosthetic device to a vertebrae;

25. A dilator, comprising:
a body portion having a substantially oval cross-section defining a length x and a width y.

26. The dilator of claim 25, wherein the ratio between x and y is 2:1.

27. The dilator of claim 25, wherein the ratio between x and y is 2:1.5.
